(19) United States
(12) Patent Application Publication
(10) Pub. No.: US 2006/0246137 A1
(43) Pub. Date: Nov. 2, 2006

(54) Complex matrix for biomedical use

(76) Inventors: Laurence Hermitte, Luynes (FR); Olivier Benoit, Annecy (FR)

Correspondence Address: YOUNG & THOMPSON, 745 SOUTH 23RD STREET, 2ND FLOOR, ARLINGTON, VA, 22202, US

(21) Appl. No.: 10/565,442

(22) PCT Filed: Jul. 30, 2004
(86) PCT No.: PCT/FR04/02052

§ 371 Date: Jul. 5, 2006

(30) Foreign Application Priority Data
Jul. 30, 2003 (FR) 0309401

(51) Int. Cl. A61K 9/14

(52) U.S. Cl. 424/486; 424/488

(57) ABSTRACT

A complex matrix is constituted by at least one biocompatible polymer of natural origin, cross linked and to which are grafted small chains of a molecular weight less than 50,000 Da with an amount of grafting of 10 to 40%, as well as a process for preparation of a biocompatible matrix that is partly degradable, constituted by at least one polymer of natural origin, consisting: on the one hand, grafting small chains of molecular weight less than 10,000 Da, with a grafting amount of 10 to 40%, on the other hand, cross linking the principal polymer chains to create a homogeneous matrix.

The present invention relates to a biocompatible matrix, constituted by at least one polymer of natural origin, strongly functionalized, permitting the replacement of biological fluids, the separation of tissues or tissue increase. The matrix of the present invention is characterized by a long persistence in vivo, obtained by retarding its chemical, biological and mechanical degradation.

The present invention provides a process and compositions in the form of a complex matrix of at least one polymer of natural. origin, to obtain medical (pharmacologically active) devices adapted to increase the tissue separation or viscosupplementation, totally biodegradable but characterized by a long persistence in vivo.

The injection of a viscoelastic solution is often envisaged to replace the natural synovial liquid which, in arthrosic patients, can no longer ensure chondroprotective functions, lubrication and absorption of shocks given a reduction of the quantity of the molecular weight of the constituent glycosaminoglycanes. These products are rapidly eliminated from the synovial pocket.

The tissue increase is desired both in the case of therapeutic applications and for cosmetic purposes.

In the case of therapeutic applications, certain tissues require being enlarged to ensure their function; this can be the case of vocal cords, the esophagus, the urethral sphincter, other muscles . . . .

The patients can have recourse to aesthetic surgery for overcoming wrinkles, masking scars, increasing the lips . . . . But, in addition to the high cost associated with this practice, the drawbacks are numerous, because it is an invasive and risky procedure. Injection of materials adapted to increase tissue is a widely used method. The hypodermic needles used as medical device have the advantage of being easy to use, precise, and constituting a non-invasive method.

The injectable materials available on the market are products either permanent or biodegradable.

Non-Resorbable Permanent Products

There exist two approaches for using non-resorbable products: the injection of silicone or a suspension of solid particles in a vector solution.

The injection of silicone has been widely used. However, given the undesirable long-term effects (nodules, ulcers of the skin), this method is more and more abandoned [Edgerton et al. “Indications for and pitfalls of soft tissue augmentation with liquid silicone”. Plast. Reconstr. Surg, 58:157-163 (1976)].

The injection of solid microparticles also permits an increase of permanent tissue.

U.S. Pat. No. 5,344,452 discloses the use of a pulverulent solid, constituted by small particles, of a diameter comprised between 10 μm and 200 μm, and having a very smooth surface. Artecoll® and Arteplast®, products of commerce, are constituted by a suspension of microspheres of polymethacrylate in a collagen solution.

EP-A-1 091 775 proposes a solution of fragments of methacrylate hydrogel in a solution of hyaluronate. The particles of silicone, ceramics, carbon or metals (U.S. Pat. No. 5,451,406, U.S. Pat. No. 5,792,478, U.S. 2002-151466), the fragments of polytetrafluoroethylene, of glass or synthetic polymers (U.S. 2002-025340), and balls of collagen have also been used but the results have been disappointing, given the secondary reactions, and the biological degradation and the migration of the residual products. Thus, the particles have least one of these drawbacks: a too great diameter or an irregular shape, which makes the particles cling to each other, which can render the injection difficult through a fine needle, the too fragile particles can break during injection, the injection of too small particles leads to rapid digestion by the macrophages and other constituents of the lymphatic system, the injected particles can move and not adhere to the environmental cells.

The permanent character of these products accordingly leads to major drawbacks: the risk of activation of macrophages, the migration of the synthetic fragments constituting the product or the appearance of granuloma which can require the injection of steroids, or even an excision. Moreover, this type of product does not permit retouching if necessary.

Among the degradable biological materials, can be mentioned solutions of collagen or of cross-linked hyaluronic acid.

Collagen Corporation has developed a preparation based on collagen cross-linked with glutaraldehyde (U.S. Pat. No. 4,582,640). This product is digested by the enzymatic or biochemical route, by macrophages, eliminated by the lymphatic system, and hence rapidly degraded. Repeated treatments are accordingly necessary.

U.S. Pat. No. 5,137,875 claims the use of aqueous suspensions or solutions of collagen containing hyaluronic acid, but this product cannot constitute a solution for long term treatment.

EP 0 466 300 proposes the injection of a viscoelastic gel comprised by a matrix dispersed in a liquid phase, the two phases being composed by hylan, hyaluronate of high molecular weight of animal origin, cross linked and extracted.

The hyaluronic acid esters and the cross linked derivatives of hyaluronic acid have been developed for the purpose of increasing the time of absorption of this glycosaminoglycane and hence obtaining greater residence times. Among such products adapted for cosmetic use, can be cited Restylane®, a biphasic gel constituted by a fluid phase (non-cross-linked hyaluronate), and a very cross linked phase. If the intermolecular or intramolecular linkages of polysaccharides or esters of acid polysaccharides are used for numerous applications, for example the prevention of post-surgical adherence (EP 0 850 074, U.S. Pat. No. 4,851,521, EP 0 341 745), these products cannot constitute a long persisting effect given the high level of enzymatic degradation and the low lifetime of the ester linkages which, contrary to ether linkages, are degradable in physiological environments (U.S. Pat. No. 4,963,666).

So as to increase the persistence of the matrix, it can be noted that the tendency is to use polymers of high molecular weight or to increase the degree of cross linkage. But if the cross linkage increases in a substantial manner the lifetime of the product, the manipulation of these highly cross-linked gels, and hence very constrained, is very delicate because the other sites of the polymer not protected by a cross linkage are mechanically and chemically rendered fragile and more susceptible to being attacked.

Moreover, a large increase of the degree of cross linkage can lead to products that are difficult to inject.

EP 0 749 982 proposes grafting an antioxidant to a matrix with a low rate of grafting.

It thus appears clearly that the existing materials do not provide an ideal solution, and the search for new products for the increase of tissue, the separation of the tissues or the viscosupplementation, continues, with the aim of identifying highly biocompatible materials, easily used in the field of clinical use, having a lifetime such that this product disappears when its function is no longer needed, but sufficient to limit the medical and surgical interventions.

### SUMMARY OF THE INVENTION

Although the conditions for increase, tissue separation and viscosupplementation have been known for many years, and numerous solutions have been proposed for therapeutical and cosmetic applications, the present invention provides a process and proposes new compositions permitting the medical device to be effective long term without secondary effects. These same compositions can also prove to be useful to constitute vectors for active pharmacological substances.

The principle of the present invention is based on the occupation of a large number of sites of the polymeric chains to retard chemical and enzymatic attacks directly on the principal chain of the polymer. The grafting of small molecules coupled with cross linkage leads to increase of the density of the matrix, and hence the time necessary for it to degrade, whilst limiting its agility induced by a too great degree of cross linkage. The coupling of two types of functionalization, reticulation and grafting, also permits increasing the ease of use of a matrix adapted to be injected by recourse to a matrix which has the same number of sites occupied on the principal chain of the polymer but whose degree of cross linkage is greater. The effect permitting the long persistence of the composition can be amplified if the grafted molecules have antioxidant properties. Antioxidant agents can also be dispersed in the matrix. The use of cellulosic derivatives or other polymers naturally absent in the human being to constitute the product, also permits retarding the degradation of the matrix given the lack of specific hydrolases.

In the context of the present invention, the word “site” designated all the points on the polymer chain adapted to be attacked; it can be a matter of pendant functional groups such as hydroxy or carboxy groups or a chain such as ether linkages.

The effect of long persistence of the medical device permits spacing the medical interventions and hence improving the quality of the life of the patients.

Another object of the present invention is to provide a same composition containing one or several therapeutically active molecules.

### DETAILED DESCRIPTION OF THE INVENTION

The present invention provides a biocompatible complex monophasic matrix with long persistence, comprised by at least one highly functionalized polymer of natural origin. By long persistence, is meant an in vivo lifetime greater than that of a product having an identical degree of functionalization but obtained by another process than that of the present invention, characterized most often by a single cross linkage.

The substance adapted for viscosupplementation or tissue augmentation is comprised by at least one polymer of a molecular weight greater than 100,000 Da, selected from polysaccharides such as hyaluronic acid, chondroitine sulfate, keratane, keratane sulfate, heparin, heparin sulfate, cellulose and its derivatives, xanthanes and alginates, proteins, or nucleic acids, this polymer being highly functionalize by grafting of small chains and a cross linkage permitting the creation of a matrix. By matrix is meant a three-dimensional network constituted by polymers of biological origin doubly functionalized by cross linking and grafting.

The cross linking agent can be selected particularly from di- or polyfunctional epoxides, for example 1,4-butanediol diglycidyl ether (also called 1,4-bis (2,3-epoxypropoxy)butane), 1-(2,3-epoxypropyl)2,3-epoxy cyclohexane and 1,2-ethanediol diglycidyl ether, the epihalohydrins and divinylsulfone.

The degree of reticulation, defined as the ratio between the number of moles of reticulant ensuring the linkage of the chains of the polymer and the number of moles of structures of the polymer, is comprised between 0.5 and 25% in the case of injectable products, from 25 to 50% in the case of solids.

So as to increase the steric size and the density of the matrix, and hence the time necessary for the product to be degraded by chemical and biochemical action, small chains can be grafted by ionic linkages or in a covalent fashion, preferably by etherification, onto the matrix. These grafted chains will occupy a large number of sites on the matrix, which permits increasing substantially the lifetime of the product without modifying the mechanical or rheological character of the polymer constituting the matrix. To the mechanical protection is added a biological and chemical protection constituted by “lures”.

The chains grafted on the functional groups of the hydroxy or carboxy type probably protect on the one hand directly these functional groups having reacted, and on the other hand indirectly the other sites detectable by steric hindrance.

The grafted chains and the polymers of natural origin of small size comprise more available attackable sites than the sites masked by the matrix, or polymers not recognized by the enzymes of the organism. In this latter case, it can be a matter of cellulosic derivatives or of derivatives of other biopolymers not naturally present in the human body which will not be degraded by the enzymes of the organism, but will be sensitive to attack by the free radicals and other reactive radicals. It can for example be a matter of carboxymethylcellulose.

The grafted chains can moreover be unpolymerized chains having antioxidant properties or properties to inhibit the reactions of degradation of the polymer matrix. It can for example be a matter of vitamins, enzymes or cyclic molecules.

The amount of grafting which is defined as the ratio between the number of moles of grafted molecules or the number of moles of the grafted polymer and the number of moles of the structure of the cross linked polymer or polymers, is comprised between 10 and 40%.

The grafting of the chains of small size, which is to say of a size less than 50,000 Da, and preferably of the order of 10,000 Da or less, on numerous sites of the polymer matrix, permits preserving the injectable character of the final product because the amount of reticulation is not increased, whilst the presence of these grafted chains prevents the attack of the matrix by the environmental medium and ensures a longer persistence of the product after injection.

The grafted molecules can be grafted by covalent linkage to the principal chains, directly for example by esterification or etherification of the hydroxy or carboxy groups of by means of a bi- or polyfunctional molecule selected from epoxids, epihalohydrins or divinylsulfone.

Those skilled in the art will easily understand that such a process of functionalization has significant advantages relative to a simple cross linkage.

The grafting and cross linkage can take place at the same time, or the grafting can precede the cross linkage, or vice versa.

So as to retard degradation by free radicals, a molecule having antioxidant properties may also be dispersed in the strongly functionalized matrix.

For example, vitamin C, a slender hydrosoluble molecule having antioxidant properties, can be used in the case of non-inflamed tissues to avoid the oxidation of the organic macromolecules, to capture the free radicals, but also to stimulate the synthesis of the extracellular matrix, particularly of collagen. This effect can be particularly interesting in the case of dermatological and cosmetic applications, to improve the elasticity of the skin.

Vitamin A, which has numerous advantages (antioxidant action, influence on the development of tissues and participation in the treatment of the skin) could also be dispersed in this highly modified matrix which, by its density, would permit progressive release of the active pharmacological agent.

Melatonin, which would be released in a very small quantity, is a powerful antioxidant agent and regenerator of the skin and defender of the immune system which could also be dispersed in the matrix.

So as to retard enzymatic degradation, the use of polymers not naturally available in the human body such as cellulosic derivatives, particularly carboxymethylcellulose, is recommended in the composition of matrices of the present invention, given the absence of specific hydrolases of these polymers.

As a result, the long persistence effect of the products of the present invention is obtained by greatly increasing the steric hindrance, by blocking a very large number of “attackable” sites biologically and chemically without rendering the other sites fragile, thanks to the use of the grafting of short chains and a quantity of cross linkage which remains fairly low compared to other products presently on the market.

Moreover, this type of functionalization permits for a number of identical occupied sites on the principal chains of the constituent polymer of the matrix, an injectability facilitated relative to that of gels modified by cross linkage alone.

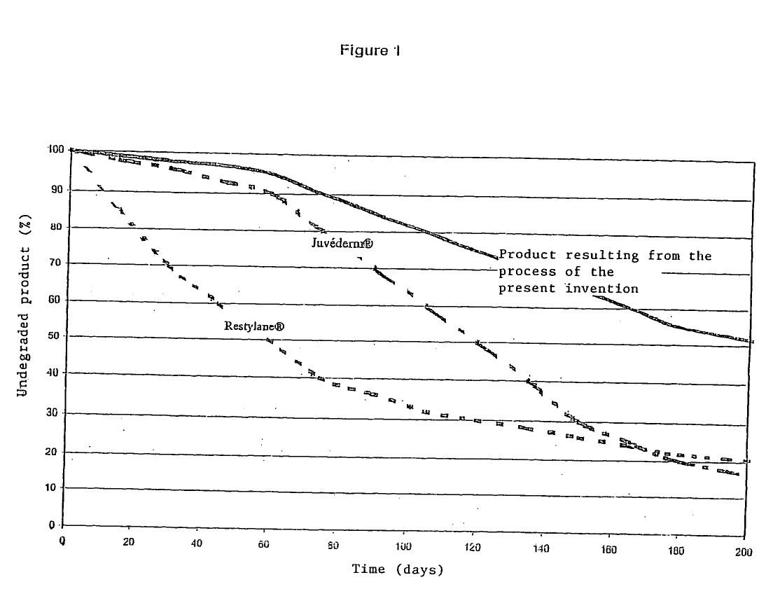

FIG. 1 shows the much slower degradation as a function of time, of injectable products according to the present invention, and two products available on the market, Juvéderm® and Restylane® (composition of polysaccharide gel of U.S. Pat. No. 5,827,937).

The invention also relates to a complex matrix constituted by at least one biocompatible polymer of natural origin, cross linked and to which are grafted chains of molecular weight less than 50,000 Da with a quantity of grafting of 10 to 40%.

The biocompatible polymer of natural origin constituting the matrix is preferably selected from polysaccharides such as hyaluronic acid, chondroitine sulfate, keratane, keratane sulfate, heparin, heparane sulfate, cellulose and its derivatives, xanthanes and alginates, proteins, or nucleic acids.

According to a preferred embodiment, the biocompatible polymer of natural origin is a polymer not naturally present in the human body, such as a cellulosic derivative, a xanthane or an alginate, which is cross linked with at least one polymer naturally present in the human body selected from polysaccharides such as hyaluronic acid, chondroitine sulfate, keratane, keratane sulfate, heparin, heparin sulfate, xanthanes and alginates, proteins or nucleic acids.

Preferably, the amount of cross linkage, defined as the ratio between the number of moles of reticulating agent ensuring the linkage of the polymer chains and the number of molds of polymer structure, is comprised between 0.5 and 50%, in particular between 0.5 and 25% in the case of injectable products, and between 25 and 50% in the case of solid products. The cross linking agent ensuring the linkage of the chain can be provided by a bi- or polyfunctional molecule selected from epoxydes, epihalohydrines and divinylsulfone.

The matrix can contain antioxidant agents, vitamins or other pharmaceutically active dispersed agents.

The invention also relates to the use of the matrix defined above to replace, fill or supplement a biological fluid or tissues.

The invention also relates to a process to obtain a biocompatible matrix which is partly biodegradable, constituted by at least one polymer of natural origin, characterized in that it consists:
on the one hand in grafting small chains of molecular weight less than 50,000 Da with a grafting quantity of 10 to 40%,
on the other hand, cross linking the principal chains of the polymer to create a homogeneous matrix.

### EXAMPLES

Examples are provided to illustrate the invention, but in no case do they limit the scope of the invention.

### First Series of Examples (Examples 1 to 3)

### Example 1—(Cross Linkage)

150 mg of sodium hyaluronate (M.W.=2×10⁶ Da) and 50 mg of carboxymethylcellulose (M.W.=2×10⁵ Da) are added to 6 ml of 0.5% soda. The whole is homogenized in a mixture until a transparent solution is obtained. 10 μl of 1,4-butanediol diglycidyl ether (BDDE) are then added to the solution and the whole is mixed for 12 hours at 20° C. The pH is adjusted to physiological pH. The obtained matrix is then dialyzed for 24 hours (regenerated cellulose, limit of separation, M.W.=12,000-14,000) against a solution of phosphate buffer at pH 7 (gel 1).

### Example 2—(Cross Linkage)

150 mg of sodium hyaluronate (M.W.=2×10⁶ Da) and 50 mg of carboxymethylcellulose (M.W.=2×10⁵ Da) are added to 6 ml of 0.5% soda. The whole is homogenized in a mixture to obtain a transparent solution. 20 μl of 1,4-butanediol diglycidyl ether (BDDE) is then added to the solution and the whole is mixed for 12 hours at 20° C. The pH is readjusted to physiological pH. The obtained matrix is then dialyzed for 24 hours (regenerated cellulose, limit of separation, M.W.=12,000-14,000) against a phosphate buffer solution at pH 7 (gel 2).

### Example 3—(Cross Linkage and Grafting)

150 mg of sodium hyaluronate (M.W.=2×10⁶ Da) and 50 mg of carboxymethylcellulose (M.W.=2×10⁵ Da) are added to 6 ml of 0.5% soda. The whole is homogenized in a mixture until a transparent solution is obtained. 20 μl of 1,4-butanediol diglycidyl ether (BDDE) is then added to the solution and the whole is mixed for 8 hours at 20° C. 40 mg of benzyl hyaluronate (esterified to 75%, M.W.=10⁴ Da) are added and mixed for 2 hours at 20° C. 10 mg of vitamin C is then added and incorporated in the viscous matrix. The pH is adjusted to physiological pH. The whole is then mixed for 2 hours. The obtained matrix is then dialyzed for 24 hours (regenerated cellulose, limit of separation, M.W.=12,000-14,000) against a solution of phosphate buffer at pH 7 (gel 3).

Calculation of the Amount of Grafting:

$$\begin{matrix}Quantity of grafting = \frac{( ( {m}_{vitC} / {M}_{vitC} + ( {m}_{HAbenzyl} / {M}_{HAbenzyl} ) )}{( ( {m}_{HA} / {M}_{HA} ) + ( {m}_{CMC} / {M}_{CMC} ) )} \\ 0.246 ( which is to say 24.6 \% )\end{matrix}$$

wherein:
m: weight in g
M: molecular weight of the polymer unit in g/mol
Vit C: vitamin C
HA: hyaluronate
HAbenzyl: benzyl hyaluronate
CMC: carboxymethylcellulose

The amount of grafting, calculated by supposing that the carboxylic functions are all in the form of sodium salt and that the carboxymethylcellulose has a quantity of substitution of 0.9, is 24.6%.

Rheological studies have shown a slower decrease of these properties for the gel of example 2 (gel 2) than for that of example 1 (gel 1) when these gels are held at 37° C. Although an in vivo study has not been carried out to date, the degradation of gel 2 is probably slower than that of gel 1, which itself must be degraded less rapidly than a synthesized gel according to the same process but comprised exclusively of sodium hyaluronate. This result is suggested by the data concerning the in vivo lifetime of the unreticulated carboxymethylcellulose, compared to that of unreticulated sodium hyaluronate injected in the same concentration and having a comparable molelcular weight.

Gel 2 has a lifetime greater than that from the first example thanks to a degree of cross linkage twice as high.

The number of sites occupied in the gel of example 3 (gel 3) is at least equal to that of gel 2 and the decrease in the viscosity of gel 3 in the course of time is slower than that of gel 2 (when these gels are held at 37° C.).

### Second Series of Examples (Examples 4 to 7)

### Example 4—(Cross Linkage)

1 g of sodium hyaluronate (M.W.=2×10⁶ Da) is placed in 10 ml of a soda solution at 1%. The whole is homogenized with a mixture until the solution becomes transparent. 100 μl of 1,4-butanediol diglycidyl ether (BDDE) is then added and the whole is again mixed for 2 hours at 50° C. The solution is adjusted to physiological pH and the volume is readjusted to 50 ml with a phosphate buffer. The obtained matrix is then dialyzed for 24 hours (regenerated cellulose, limit of separation, M.W.=12,000-14,000) against a phosphate buffer solution at pH 7 (gel 4).

### Example 5—(Cross Linkage)

1 g of sodium hyaluronate (M.W.=2×10⁶ Da) is placed in 10 ml of a 1% soda solution. The whole is homogenized with a mixture until the solution becomes transparent. 130 μl of 1,4-butanediol diglycidyl ether (BDDE) is then added and the whole is again mixed for 2 hours at 50° C. The solution is adjusted to physiological pH and the volume is readjusted to 50 ml with a phosphate buffer. The obtained matrix is then dialyzed for 24 hours (regenerated cellulose, limit of separation, M.W.=12,000-14,000) against a phosphate buffer solution of pH 7 (gel 5).

### Example 6—(Cross Linkage)

0.8 g of sodium hyaluronate (M.W.=2×10⁶ Da) and 0.2 g of carboxymethylcellulose (M.W.=3×10⁵ Da) are placed in 10 ml of a 1% soda solution. The whole is homogenized with a mixer until the solution becomes transparent. 130 μl of 1,4-butanediol diglycidyl ether (BDDE) is then added and the whole is again mixed for 2 hours at 50° C. The solution is adjusted to physiological pH and the volume is readjusted to 50 ml with a phosphate buffer. The obtained matrix is then dialyzed for 24 hours (regenerated cellulose, limit of separation, M.W.=12,000-14,000) against a phosphate buffer solution of pH 7 (gel 6).

### Example 7—(Cross Linkage and Grafting)

0.8 g of sodium hyaluronate (M.W.=2×10⁶ Da) and 0.2 g of carboxymethylcellulose (M.W.=3×10⁵ Da) are placed in 10 ml of a 1% soda solution. The whole is homogenized with a mixer until the solution becomes transparent. 130 μl of 1,4-butanediol diglycidyl ether (BDDE) are then added and the whole is mixed for 1 hour 20 minutes at 50° C. 0.2 g of heparin (M.W.=3×10³ Da) diluted in 4 ml of 0.5% soda solution is then added to the gel in the course of formation and the whole is again mixed. The mixture is brought to physiological pH and the volume is readjusted to 50 ml with a phosphate buffer. The obtained matrix is then dialyzed for 24 hours (regenerated cellulose, limit of separation, M.W.=12,000-14,000) against a phosphate buffer of pH 7 (gel 7).

Computation of Amount of Grafting:
Amount of grafting=(mₕₑₚₐᵣᵢₙ/Mₕₑₚₐᵣᵢₙ)/((m_{HA}/M_{HA})+(m_{CMC}/M_{CMC}))=10.3%
wherein:
m: weight in g
M: molecular weight of the polymer unit in g/mol
HA: hyaluronate
CMC: carboxymethylcellulose

The quantity of grafting, calculated by supposing that half the ionizable functions are in the form of sodium salt and that the carboxymethylcellulose has a substitution amount of 0.9, is 10.3%.

Moreover, a process has been set forth to quantify the injectability of the different gels obtained in examples 1 to 7. This process uses the measurement of the force necessary for the ejection of the different gels obtained through a needle of type 27 G. Each obtained gel is placed in a syringe of 1 ml whose outlet is provided with a needle of type 27 G. The syringe is held vertical by a carrier and a weight is then applied to the piston of the syringe, at a constant speed defined by the user. A detector measures the force necessary to eject the product. In the first series of examples, the speed of ejection is 75 mm/min and in the second series of examples, the speed of ejection is 15 mm/min.

The values of the force of ejection measured for the gels of examples 1 to 7 is given in tables 1 and 2 hereafter.

TABLE 1

| | Force of ejection |
|---|---|
| Gels | V = 75 mm/min |
| 1 (cross linkage) | 20N +/− 4N |
| 2 (cross linkage) | 32N +/− 4N |
| 3 (cross linkage and | 25N +/− 4N |
| grafting) | |

According to the results given in the table, for an equivalent amount of cross linkage, the cross linked and grafted gels according to the invention have a force of ejection less (and hence a better injectability) than that of the cross linked gels (comparison of gels of example 2 and example 3).

TABLE 2

| | Force of ejection |
|---|---|
| Gels | V = 15 mm/min |
| 4 (cross linkage) | 14N +/− 4N |
| 5 (cross linkage) | 23N +/− 4N |
| 6 (cross linkage) | 26N +/− 4N |
| 7 (cross linkage and | 24N +/− 4N |
| grafting) | |

As previously observed, an increase in the amount of cross linkage leads to increase of the force necessary to eject the product (comparison of gels 4 to 6). At an identical amount of cross linkage, this injectability is more difficult for cross linked gels HA/CMC. But if the injectability is higher, the persistence of these gels must also be longer. The last example (comparison of gels 6 and 7) emphasizes the fact that the grafting of small chains of heparin permits decreasing the force necessary for ejection whilst protecting the cross linked matrix, by steric hindrance and by the biological properties of this polymer.

1-11. (canceled)

12. Complex matrix constituted by at least one biocompatible polymer of natural origin, cross linked with a cross linking agent of a bi- or polyfunctional molecule selected from epoxydes, epihalohydrines and divinylsulfone and on which are grafted chains of molecular weight less than 50,000 Da, selected from polymers of natural origin of small size, preferably cellulosic derivatives or other derivatives of biopolymers not naturally present in the human body and/or non-polymeric chains having antioxidant properties or properties for inhibiting reactions of degradation of said matrix, preferably vitamins, enzymes or molecules comprising one or several cycles, with a quantity of grafting, defined as being the ratio between the number of moles of grafted molecules and the number of moles of units of the polymer, from 10 to 40%.

13. Matrix according to claim 12, in which the biocompatible polymer of natural origin is selected from hyaluronic acid, chondroitine sulfate, keratane, keratane sulfate, heparin, heparin sulfate, cellulose and its derivatives, xanthanes and alginates, proteins, or nucleic acid.

14. Matrix according to claim 12, in which the biocompatible polymer of natural origin is a polymer not naturally present in the human body such as a cellulosic derivative, a xanthane or an alginate which is cross linked with at least one polymer naturally present in the human body selected from hyaluronic acid, chondroitine sulfate, keratane, keratane sulfate, heparin, heparane sulfate, xanthanes and alginates, proteins or nucleic acids.

15. Matrix according to claim 12, in which the amount of cross linkage, defined as the ratio between the number of moles of the cross linking agent ensuring the linking of the polymer chains and the number of moles of units of the polymer, is comprised between 0.5 and 50%, in particular between 0.5 and 25% in the case of injectable products, and between 25 and 50% in the case of solid products.

16. Matrix according to claim 12, containing antioxidant agents, vitamins and other dispersed pharmacologically active agents.

17. Matrix according to claim 12, containing vitamins or other dispersed pharmacologically active agents.

18. A method to separate, replace, fill or supplement a biological fluid or tissues comprising a step of applying an effective amount of a matrix according to claim 12.

19. Process for the preparation of a partly biodegradable biocompatible matrix constituted by at least one polymer of natural origin, characterized in that it consists in:
on the one hand grafting small chains of molecular weight lower than 50,000 Da with an amount of grafting of 10 to 40%, the small chains being selected from polymers of natural origin of small size, preferably cellulosic derivatives or derivatives of other biopolymers not naturally present in the human body and/or unpolymerized chains having antioxidant properties or properties of inhibiting reactions of degradation of said matrix, preferably vitamins, enzymes and molecules comprising one or several units,
on the other hand cross linking the principal chains of the polymer to create a homogeneous matrix, with the help of a cross linking agent which is a bi- or polyfunctional molecule selected from epoxydes, epihalohydrines or divinylsulfone.

20. Matrix according to claim 13, in which the biocompatible polymer of natural origin is a polymer not naturally present in the human body such as a cellulosic derivative, a xanthane or an alginate which is cross linked with at least one polymer naturally present in the human body selected from hyaluronic acid, chondroitine sulfate, keratane, keratane sulfate, heparin, heparane sulfate, xanthanes and alginates, proteins or nucleic acids.

21. Matrix according to claim 13, in which the amount of cross linkage, defined as the ratio between the number of moles of the cross linking agent ensuring the linking of the polymer chains and the number of moles of units of the polymer, is comprised between 0.5 and 50%, in particular between 0.5 and 25% in the case of injectable products, and between 25 and 50% in the case of solid products.

22. Matrix according to claim 14, in which the amount of cross linkage, defined as the ratio between the number of moles of the cross linking agent ensuring the linking of the polymer chains and the number of moles of units of the polymer, is comprised between 0.5 and 50%, in particular between 0.5 and 25% in the case of injectable products, and between 25 and 50% in the case of solid products.

23. Matrix according to claim 13, containing antioxidant agents, vitamins and other dispersed pharmacologically active agents.

24. Matrix according to claim 14, containing antioxidant agents, vitamins and other dispersed pharmacologically active agents.

25. Matrix according to claim 15, containing antioxidant agents, vitamins and other dispersed pharmacologically active agents.

26. Matrix according to claim 13, containing vitamins or other dispersed pharmacologically active agents.

27. 16. Matrix according to claim 14, containing vitamins or other dispersed pharmacologically active agents.

28. Matrix according to claim 15, containing vitamins or other dispersed pharmacologically active agents.

29. Matrix according to claim 16, containing vitamins or other dispersed pharmacologically active agents.

30. A method to separate, replace, fill or supplement a biological fluid or tissues comprising a step of applying an effective amount of a matrix according to claim 13.

31. A method to separate, replace, fill or supplement a biological fluid or tissues comprising a step of applying an effective amount of a matrix according to claim 14.
